(19) United States
(12) Patent Application Publication
(10) Pub. No.: US 2008/0147083 A1
(43) Pub. Date: Jun. 19, 2008

(54) Method and device for fixation of ophthalmic tissue

(76) Inventors: Steven D. Vold, Bentonville, AR (US); William Plauche, Sherman, TX (US)

Correspondence Address: TROP PRUNER & HU, PC, 1616 S. VOSS ROAD, SUITE 750, HOUSTON, TX, 77057-2631, US

(21) Appl. No.: 11/983,610

(22) Filed: Nov. 9, 2007

Related U.S. Application Data
(60) Provisional application No. 60/857,964, filed on Nov. 9, 2006.

(51) Int. Cl. A61F 2/16; A61F 9/007

(52) U.S. Cl. 606/107; 606/151; 623/6.12

(57) ABSTRACT

In an embodiment of the invention, a method includes using an implantable ocular clip to fix an intraocular lens to an iris, all without having to use a suture to permanently hold the lens in place.

This application claims priority to U.S. Provisional Patent Application No. 60/857,964 filed on Nov. 9, 2006 entitled METHOD AND DEVICE FOR FIXATION OF OPHTHALMIC TISSUE.

### BACKGROUND

Ocular maladies present numerous challenges to health care providers. Cataracts provide one such malady. To treat cataracts, physicians often replace the problematic natural lens of the eye with an artificial intraocular lens (IOL). IOLs may have side members, referred to as haptics, which help stabilize the lens within the eye. In some cases, a clip is connected to the haptic or lens optic by the lens manufacturer. After inserting the IOL into the eye, the physician then attempts to secure the IOL in the eye by connecting the clip to ocular tissue such as the inner iris. Doing so, however, often leads to iris chafing brought on by the interaction between the clip and the inner iris, an area of the eye that is very active and non-stationary. The chafing often leads to inflammation and shedding of iris pigment epithelial cells. These cells may then occlude natural aqueous fluid drainage channels. Hindering the drainage channels may cause undesired fluid retention in the eye, thereby increasing intraocular pressure, which is a contributing factor for glaucoma. Such chafing may also lead to other maladies such as, for example, cystoid macular edema and corneal decompensation.

Present ocular clips not only lead to chafing, they also are very limited in their utility. In other words, the clip is permanently affixed to a lens. Thus, if such a clip fails, the physician must typically replace the entire IOL instead of only replacing the faulty clip.

Thus, use of traditional ocular clips has declined in favor of advanced suturing techniques. While such suturing techniques are clinically efficacious, they are also complicated and practiced by only highly skilled physicians. The advanced suturing techniques lead to increased procedure time which can result in increased surgical complications, chances for infection, and overall cost and inconvenience to the patient. Late suture breakage, which may occur months or years after the initial suturing is performed, may also lead to a whole new set of complications including IOL dislocation and retinal detachment.

### BRIEF DESCRIPTION OF THE DRAWINGS

The accompanying drawings, incorporated in and constituting a part of this specification, illustrate one or more implementations consistent with the principles of the invention and, together with the description of the invention, explain such implementations. The drawings are not necessarily to scale, the emphasis instead being placed upon illustrating the principles of the invention. In the drawings:

FIG. 1a includes a front view of a device in one embodiment of the invention.

FIG. 1b includes a top view of a device in one embodiment of the invention.

FIG. 1c includes a top view of a device in one embodiment of the invention.

FIGS. 2-14 include front views of various embodiments of the invention.

FIG. 15 includes a front view of a device in one embodiment of the invention.

FIG. 16 includes a front view of a device in one embodiment of the invention.

FIG. 17 includes a front view of an applicator and an implant in one embodiment of the inventions.

### DETAILED DESCRIPTION

The following description refers to the accompanying drawings. Among the various drawings the same reference numbers may be used to identify the same or similar elements. While the following description provides a thorough understanding of the various aspects of the claimed invention by setting forth specific details such as particular structures, architectures, interfaces, and techniques, such details are provided for purposes of explanation and should not be viewed as limiting. Moreover, those of skill in the art will, in light of the present disclosure, appreciate that various aspects of the invention claimed may be practiced in other examples or implementations that depart from these specific details. At certain junctures in the following disclosure, descriptions of well known devices and methods have been omitted to avoid clouding the description of the present invention with unnecessary detail. Furthermore, in the following discussion and in the claims, the terms “including” and “comprising” are used in an open-ended fashion, and thus should be interpreted to mean “including, but not limited to . . . ”. Also, the term “couple” or “couples” is intended to mean either an indirect or direct mechanical, electrical, or other communicative connection. Thus, if a first component couples to a second component, that connection may be through a direct connection, or through an indirect connection via other devices and connections.

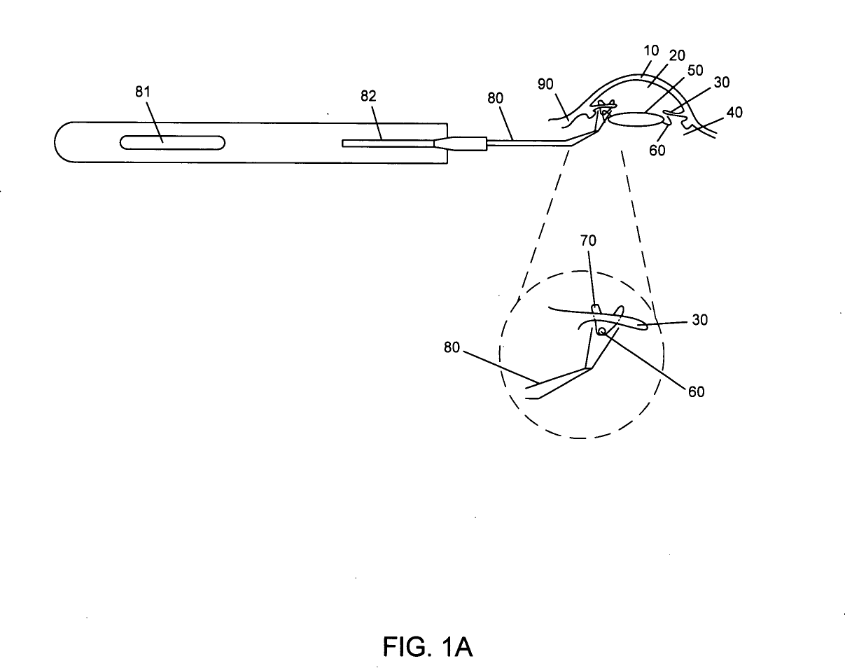

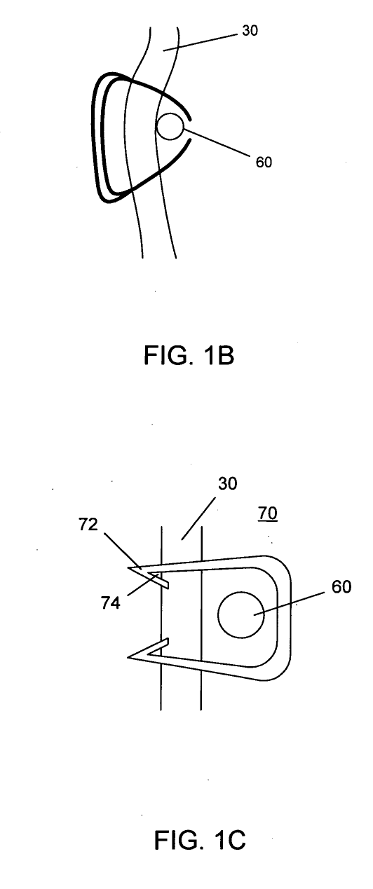

The present invention constitutes a method and apparatus for ocular fixation. As seen in FIG. 1a, ocular anatomy consists of the cornea 10, anterior chamber 20, iris 30, posterior chamber 40, and sclera 90. An IOL 50 with haptics 60 is shown in replacement of a natural lens. In one embodiment, the invention is practiced as follows. An incision is made into the anterior chamber 20. An ophthalmic clip applicator 80 and ophthalmic clip 70 are inserted through the incision into the eye. The physician abuts an open clip 70 against the anterior iris 30 and then pierces the iris with the clip.

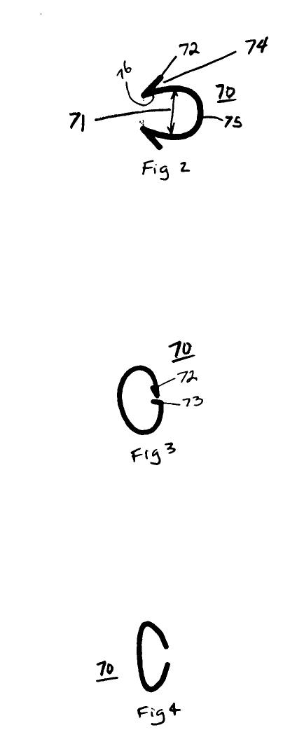

In one embodiment of the invention depicted in FIG. 2, a malleable clip 70 may have a basic “U” shape with ends 72 for piercing ocular tissue. The ends 72 may include barbs with recesses 74 for retaining the clip in ocular tissue. Due to general size limitations inherent to ophthalmic surgery, the clip may have small dimensions, including a maximum inner diameter 71 of approximately 0.05 to 0.5 mm—the approximate width of an IOL haptic. Some embodiments of the invention may include a maximum inner diameter 71 of approximately 0.15 to 0.3 mm.

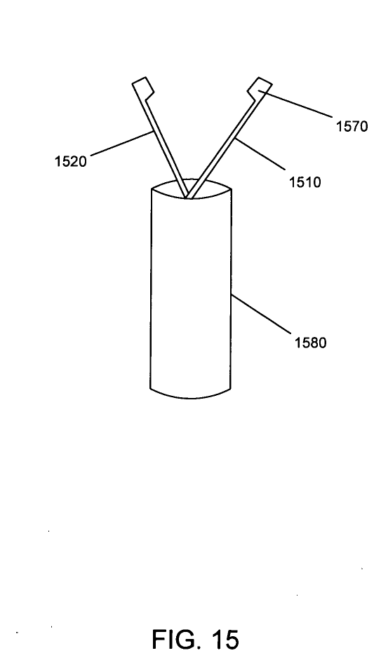

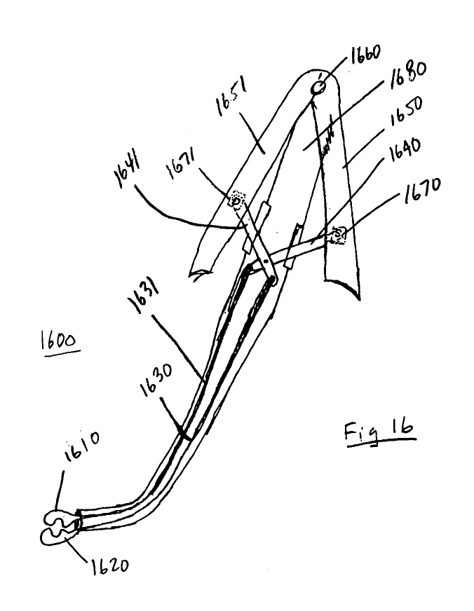

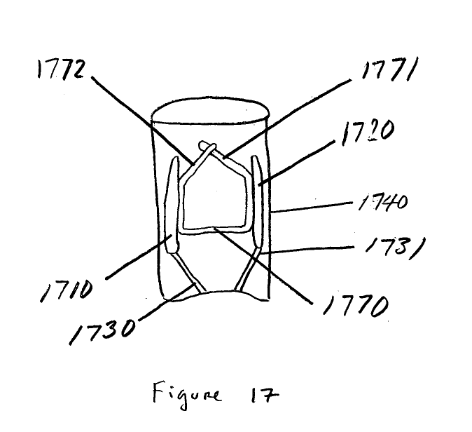

Returning to FIG. 1, after piercing the iris, the clip ends 72 are positioned across an IOL haptic 60. The physician then depresses a lever 81 on the ophthalmic clip applicator 80, thereby causing cam link 82 to constrict applicator members 1510, 1520 about the clip 1570 (FIG. 15). Thus, the physician manipulates the ophthalmic clip applicator 1580 to couple the ophthalmic clip 1570 to the haptic 60 and iris 30. The physician then removes the ophthalmic clip applicator from the eye.

In one embodiment of the invention, the clip 70 is pierced through the peripheral portion of the iris 30. By doing so, iris chafing is reduced as compared to affixing the clip 80 to the inner iris because, for example, the peripheral iris is more static and less active than the inner iris. In addition, the invention is not limited to an anterior approach. The clip may be deployed using a posterior approach whereby the haptic 60 is located in either the anterior chamber 20 or posterior chamber 40. Thus, the haptic may be held between the end portions 76 of the clip and the iris (FIG. 1b). However, the haptic may also be held between the main body of the clip 75 and the iris (FIG. 1c). Regardless, the clip 70 is deployed to hold the haptic 60 against the iris 30.

In addition, the clip 70 is not limited to affixing IOLs to the eye. The clip may be used to repair, for example, scleral tears, conjunctival tears, irregularly shaped iris tissue, or iris and corneal tissue injuries. These clips may also be used to secure both lamellar or full-thickness corneal surgery (e.g., corneal transplantation). In one embodiment of the invention, the physician inserts an ophthalmic clip applicator and an ophthalmic clip into the eye. The physician or health care provider then manipulates the ophthalmic clip applicator to couple the ophthalmic clip to a first ocular tissue and a second ocular tissue. The physician then removes the ophthalmic clip applicator from the eye. In a certain embodiment of the invention, the physician couples the ophthalmic clip to a first portion of the sclera that includes the first ocular tissue and a second portion of sclera that includes the second ocular tissue. In another embodiment of the invention, the physician couples the ophthalmic clip to a first portion of the iris that includes the first ocular tissue and a second portion of iris that includes the second ocular tissue. In yet another embodiment of the invention, the physician couples the ophthalmic clip to a first portion of the iris that includes the first ocular tissue and a first portion of sclera that includes the second ocular tissue. In short, the clip may be used to couple various portions of the eye and is therefore beneficial for numerous ophthalmic procedures.

FIGS. 1a, 15, and 16 illustrate various embodiments of a surgical clip applicator. FIG. 16 shows an applicator 1600 that comprises a housing 1680, and a handle assembly 1650, 1651, 1660 coupled to the housing 1680. The applicator 1600 also includes a jaw assembly including jaws 1610, 1620 which extend distally from the housing 1680. The jaw assembly is movable from an open position to a closed position using mechanics 1670, 1671, 1640, 1641, 1630, 1631 known to those of ordinary skill in the art. In one embodiment of the invention, a clip 70 (FIG. 2) is coupled to the jaw assembly in an open state. When the jaw assembly is manipulated into a closed position, the clip 70 is closed.

In one embodiment of the invention, the clip applicator 1580 may employ applicator members with cutting edges (not illustrated). Thus, the physician may first pierce ocular tissue with the cutting edges before deploying a clip that does not possess cutting edges. Applicator members dedicated for cutting ocular tissues may be used in cooperation with other applicator members dedicated to clip deployment (i.e., applicator members that do not employ cutting edges).

Other embodiments of the applicator may have similar pincher mechanisms and internal mechanics such as those found in, for example, Flexline™ Vitroretinal instruments from Medtronic. As those of ordinary skill in the art will appreciate, such applicators have similar ergonomic designs and mechanics so as to be readily adoptable by physicians. U.S. Pat. No. 5,868,761 discloses a representative applicator. More specifically, a clip applicator may include a handle housing formed from a pair of housing halves and secured together in a conventional manner. The handle housing may enclose a pair of handle members which are pivotable about a pivot point at the proximalmost point of the handle housing. An elongated body portion may extend from the handle housing and terminate in a jaw assembly for crimping clips upon actuation of the handle members. With reference to the handle housing, the handle members include pivot holes which are positioned about a pivot post on the handle housing halves. Pivot post, along with post members, which fit into holes, secures the housing in a snapfit-type arrangement, although other suitable means for securing the handle halves together in a conventional manner is acceptable. The handle housing halves include boss members which facilitate assembly of the components positioned within handle housing, and define a path of travel for several of the components within the handle portion. Located within the handle housing may be a cam link, which serves to advance the channel assembly to close the jaw members towards each other to crimp a clip positioned there between. The cam link may include a pair of angled slots, into which fit pins of handle members, so that as handle members are closed, pins ride within slots to drive the cam link in a distal direction. Releasing the handles permits a compression spring to drive the cam link in a proximal direction, retracting channel assembly from the jaw assembly to open the jaw members to permit the next clip in the series of clips to be fed between the jaw members. The feeding process is accomplished by a feed spring which urges a spring guide in a distal direction to advance a pusher rod, which extends through the cam link, into the elongated body portion. The pusher rod abuts against an indicator, to urge the indicator in the distal direction. The indicator abuts a proximal end of pusher nose, which in turn abuts against the series of clips to urge the clips in a distal direction and into position between the jaw members. Of course in other embodiments of the invention, the applicator may be as simple as conventional forceps that may be manipulated to deploy the implantable device in the eye.

Turning to FIG. 3, an additional embodiment of the present invention is illustrated. An ophthalmic clip 70 has first 72 and second ends 73. The first end forms a cutting surface for piercing ocular tissue. The second end abuts the first end. As seen in FIG. 17, the clip 1770 may be resilient whereby in a relaxed state, the ends 1771, 1772 abut one another. The clip 1770, housed within an applicator 1740, may be deployed into the eye. The clip 1770 may then be positioned outside the applicator 1740. Force may be exerted by the applicator extensions 1730, 1731 in an outward direction, thereby separating clip ends 1771, 1772 from one another in a stressed state. The clip 1770 may then be positioned to couple, for example only, a haptic to the iris. The applicator extensions 1730, 1731 may then be relaxed and the clip 1770 returned to its unstressed state. In the alternative, the ends of a clip may abut one another only when compressed. Of course, in alternative embodiments of the invention the two ends are separated by a small space once implanted in the eye. A person of ordinary skill in the art will appreciate that the present invention is not limited to having only one or two ends.

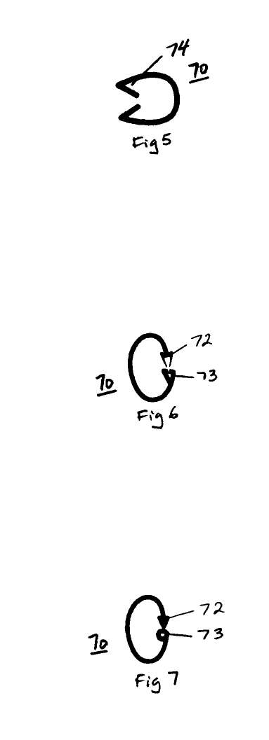

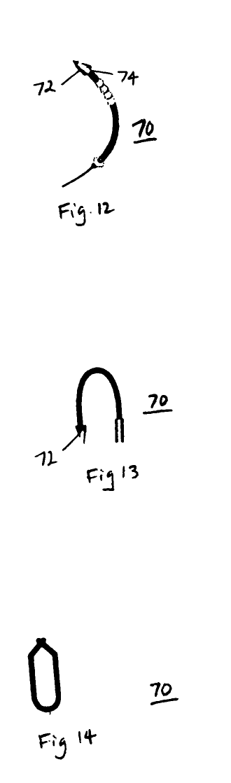

FIG. 4 discloses a barbless clip. FIG. 5 discloses a barbed clip with barbs facing inward. FIGS. 6, 7, 10, and 11 disclose clips with receptacles 73 for coupling to cutting ends 72. For example, FIG. 6 may function in a manner similar to a “zip tie” wherein the shaft portion has ledges that allow for graduated advancements of the shaft through the orifice 73. FIG. 11 may include a body 75 constructed of a suture like material such as, for example, nylon or any other biocompatible, flexible, suture-like material. FIG. 9 discloses an embodiment of the invention whereby each end 72, 73 of the clip 70 comprises a cutting edge. Once the ends penetrate ocular tissue, the recesses 74 secure the clip and prevent the ends from withdrawal from the tissue. In this embodiment, the ends need not abut, overlap or even finally reside near one another. In another embodiment of the invention, only one end 74 pierces ocular tissue. For example, one end 72 might pierce the iris from the anterior side, and then pierce the optic of an IOL while the other end 73 remains on the anterior side of the iris. In another embodiment of the invention, one end 72 might pierce the iris from the anterior side, and then pierce the optic (i.e., device need not couple to a haptic) of an IOL. In another embodiment, one end 72 might pierce the iris from the anterior side, and then capture the haptic of an IOL. In some embodiments of the invention, the optic or haptic that is to be pierced may have predrilled holes for receiving the haptic. The optic or haptic may instead have a region comprising a more easily penetrable material for promoting piercing by the clip.

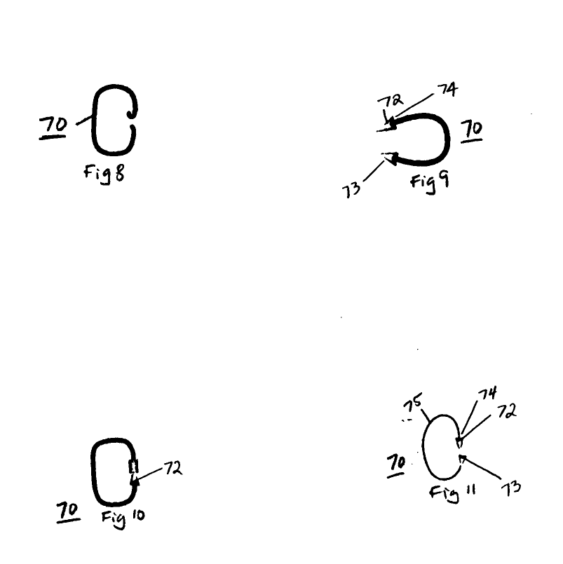

End 73 may be pointed or blunted (e.g., FIG. 8). The clips may be resilient and may be deployed into the eye in a compressed state, such as that shown in FIG. 13. The clip may then resume a noncompressed state once deployed in the eye. That noncompressed state may place the apparatus in a linear form in one embodiment of the invention. FIG. 14 illustrates another embodiment of the invention whereby a guide wire, similar to those used in PTCA procedures, is used. Thus, the physician inserts the piercing end 72 of the device through ocular tissue and/or the haptic. Once penetration has occurred, the guide wire is removed leaving the clip 70 in place. The aforementioned clips may be composed of, for example, at least one of the following materials: titanium, gold, platinum, steel, nylon, polymethyl methacrylate, polyethylene (e.g., high density polyethylene), silicone, hydrophobic or hydrophilic acrylic and polypropylene, or suture-like materials.

While the present invention has been described with respect to a limited number of embodiments, those skilled in the art will appreciate numerous modifications and variations therefrom. It is intended that the appended claims cover all such modifications and variations as fall within the true spirit and scope of this present invention.

What is claimed is:

1. An ophthalmic clip comprising:
a first clip end to couple to a first portion of an eye;
a second clip end to couple to a second portion of the eye; and
a middle clip portion coupling the first clip end to the second clip end;
wherein the ophthalmic clip is to removably couple to a previously implanted intraocular lens haptic to secure the previously implanted intraocular lens haptic to the eye.

2. The ophthalmic clip of claim 1, wherein the first clip end is to directly connect to the second clip end upon final deployment of the ophthalmic clip in the eye.

3. The ophthalmic clip of claim 1, wherein the first portion of the eye includes the peripheral iris of the eye.

4. The ophthalmic clip of claim 1, wherein the first clip end is to directly connect to the first portion of the eye and the second clip end is to directly connect to the second portion of the eye.

5. The ophthalmic clip of claim 1, wherein the first clip end is to fully traverse the first portion of the eye upon final deployment of the ophthalmic clip in the eye.

6. The ophthalmic clip of claim 1, wherein the second clip end comprises an orifice to couple to the first clip end.

7. The ophthalmic clip of claim 1, wherein the first clip end is to pass through a first portion of the intraocular lens haptic.

8. The ophthalmic clip of claim 1, wherein the first clip end is to directly connect to a first orifice of the intraocular lens haptic.

9. The ophthalmic clip of claim 1, wherein the first clip end, the second clip end, and the middle clip portion are aligned with each other in a linear manner.

10. The ophthalmic clip of claim 1, wherein the first clip end comprises a first cutting edge to cut ophthalmic tissue.

11. The ophthalmic clip of claim 1, wherein the first clip end comprises a first barb.

12. The ophthalmic clip of claim 1, wherein the first portion of the eye includes the iris of the eye and the second portion of the eye includes the anterior chamber of the eye.

13. The ophthalmic clip of claim 1, wherein the middle clip portion includes an orifice to receive a guide wire.

14. The ophthalmic clip of claim 1, wherein the ophthalmic clip is flexible.

15. A method comprising:
inserting an ophthalmic clip applicator and an ophthalmic clip into an eye that includes an intraocular lens coupled to a haptic;
manipulating the ophthalmic clip applicator to couple the ophthalmic clip to the haptic; and
removing the ophthalmic clip applicator from the eye.

16. The method of claim 1, further comprising coupling the ophthalmic clip to the iris.

17. The method of claim 1, further comprising coupling the ophthalmic clip through the iris.

18. The method of claim 3, further comprising coupling the ophthalmic clip through a peripheral portion of the iris.

19. The method of claim 3, further comprising piercing the anterior surface of the iris before piecing the posterior surface of the iris.

20. A sutureless ophthalmic implant comprising:
a first implant portion to couple to a first portion of an eye; and
a second implant portion coupled to the first implant portion, the second implant portion to couple to a second portion of the eye;
wherein the sutureless ophthalmic implant is to removably couple to an intraocular lens to secure the intraocular lens.

21. The implant of claim 20, wherein the first portion of the eye includes the iris of the eye and the second portion of the eye includes the anterior chamber of the eye.

22. The implant of claim 20, wherein the first portion of the eye includes a first portion of the iris of the eye and the second portion of the eye includes a second portion of the iris of the eye.
